(19) United States
(12) Patent Application Publication
(10) Pub. No.: US 2003/0100876 A1
(43) Pub. Date: May 29, 2003

(54) Conformable, elasticized, disposable absorbent garment

(76) Inventor: Kenneth John Molee, Suwanee, GA (US)

Correspondence Address: Christopher C. Campbell, Esq., Hunton & Williams, Suite 1200, 1900 K Street, NW, Washington, DC, 20006-1109, US

(21) Appl. No.: 09/995,755

(22) Filed: Nov. 29, 2001

(51) Int. Cl. A61F 13/15; A61F 13/20

(52) U.S. Cl. 604/385.22; 604/385.24

(57) ABSTRACT

The present invention provides an absorbent garment including a main chassis having an attachment region, an absorbent portion attached to the main chassis at an attachment region, and an elastic portion operatively associated with the main chassis. The attachment region is substantially inelastic prior to the absorbent portion being attached to the main chassis.

### FIELD OF THE INVENTION

The present invention generally relates to absorbent garments. In particular, it relates to absorbent garments having non-elastic zones.

### BACKGROUND OF THE INVENTION

Absorbent garments, such as diapers, adult incontinence products, training pants, and feminine care products, are in widespread use today.

The comfort level associated with wearing such garments is of great importance to the users of the garments. Most garments of this type include some sort of elastic material to help provide a snug, and therefore leak resistant, but still comfortable, fit.

### SUMMARY OF THE INVENTION

The invention provides a solution that can result in a more comfortable and leak preventive absorbent garment. By creating zones in the garment that are substantially inelastic, components such as absorbent cores and joining devices can be more securely attached to the garment. The substantially inelastic zones can be created by a lack of elastic material in the zone or by providing a substantially inelastic material in the zone to prevent an elastic material in the zone from stretching. This later method of providing inelastic zones can be used when a chassis of a garment contains elastic material over its entire area and the inelastic zones are created by providing a substantially inelastic material in the zones to prevent the elastic material from stretching.

The invention provides an absorbent garment including a main chassis having an attachment region, an absorbent portion attached to the main chassis at the attachment region, and an elastic portion operatively associated with the main chassis. The attachment region is substantially inelastic prior to the absorbent portion being attached to the main chassis.

The invention also provides an absorbent garment main chassis having a first carrier layer, an elastic layer, and a substantially inelastic layer. The elastic layer is substantially equal in area to the first carrier layer. The substantially inelastic layer is smaller in area than the first carrier layer and is selectively located to create areas of inelasticity.

### BRIEF DESCRIPTION OF THE DRAWINGS

FIG. 1 is an exploded view of a garment in accordance with an embodiment of the invention;

FIG. 2 is an exploded view of a garment chassis in accordance with an embodiment of the invention;

FIG. 3 is an exploded view of a garment chassis in accordance with another embodiment of the invention; and

FIG. 4 is a partial view of an assembled garment in accordance with an embodiment of the invention.

### DETAILED DESCRIPTION OF THE PREFERRED EMBODIMENTS

“Garment,” as used herein, refers to articles and garments that absorb and contain body exudates, and more specifically refers to articles and garments that are placed against or in proximity to the body of the wearer to absorb and contain the various exudates discharged from the user's body. A non-exhaustive list of examples of “absorbent articles” and garments includes diapers, diaper covers, disposable diapers, training pants, feminine hygiene products, and adult incontinence products. The invention can be used with all of the foregoing classes of absorbent articles and garments, without limitation, whether disposable or otherwise. Furthermore, the invention will be understood to encompass, without limitation, all classes and types of absorbent articles and garments, including those described above.

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Indeed, embodiments of the invention include various configurations whereby the core is folded in such a manner that the upper layer ultimately becomes the vertically highest and vertically lowest layer at the same time. Other configurations are contemplated within the context of the present invention.

The term “component” can refer, but is not limited, to designated selected regions, such as edges, corners, sides or the like; structural members, such as elastic strips, absorbent pads, stretchable layers or panels, layers of material, or the like; or a graphic. The term “graphic” can refer, but is not limited, to any design, pattern, indicia or the like.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “topsheet” and “backsheet” denote the relationship of these materials or layers with respect to the absorbent core. It is understood that additional layers may be present between the absorbent core and the topsheet and backsheet, and that additional layers and other materials may be present on the side opposite the absorbent core from either the topsheet or the backsheet.

Throughout this description, center portions, front portions, back portions, side portions and end portions are described. It is noted that one skilled in the art will know based on the teachings of this disclosure where the boundaries between such portions are. In addition, the term “front portion” is used to describe a portion of the article that is generally near the belly region of the user, the term “back portion” is used to describe a portion of the article that is generally near the back region of the user, and the term “side portions” is used to describe the portions of the article that are generally near the sides of the user.

Absorbent garments and diapers may have a number of different constructions and configurations. In each of these, it generally is the case that an absorbent core is disposed between a liquid pervious, body-facing topsheet, and a liquid impervious, exterior facing backsheet. In some cases, one or both of the topsheet and backsheet may be shaped to form a pant-like garment. In other cases, the topsheet, backsheet and absorbent core may be formed as a discrete assembly that is placed on a main chassis and the chassis is shaped to form a pant-like garment. The garment may be provided to the consumer in the fully assembled pant-like shape, or may be partially pant-like and require the consumer to take the final steps necessary to form the final pant-like shape. In the case of training pant-type garments and most adult incontinent products, the garment is provided fully formed with factory-made side seams and the garment is donned by pulling it up the wearer's legs. In the case of diapers, a caregiver usually wraps the diaper around the wearer's waist and joins the side seams manually by attaching one or more adhesive or mechanical tabs, thereby forming a pant-like structure. For clarity, the present invention is described herein only with reference to a training pant-type garment in which the topsheet, backsheet and absorbent core are assembled onto a chassis that forms a pant-like garment. Those skilled in the art will appreciate, however, that the invention may be used with other constructions.

Although the various embodiments of the invention are described in the context of a diaper, it is readily apparent and understood that this is not intended to limit the invention. The present invention may be used with any other absorbent garment having elastics incorporated therein.

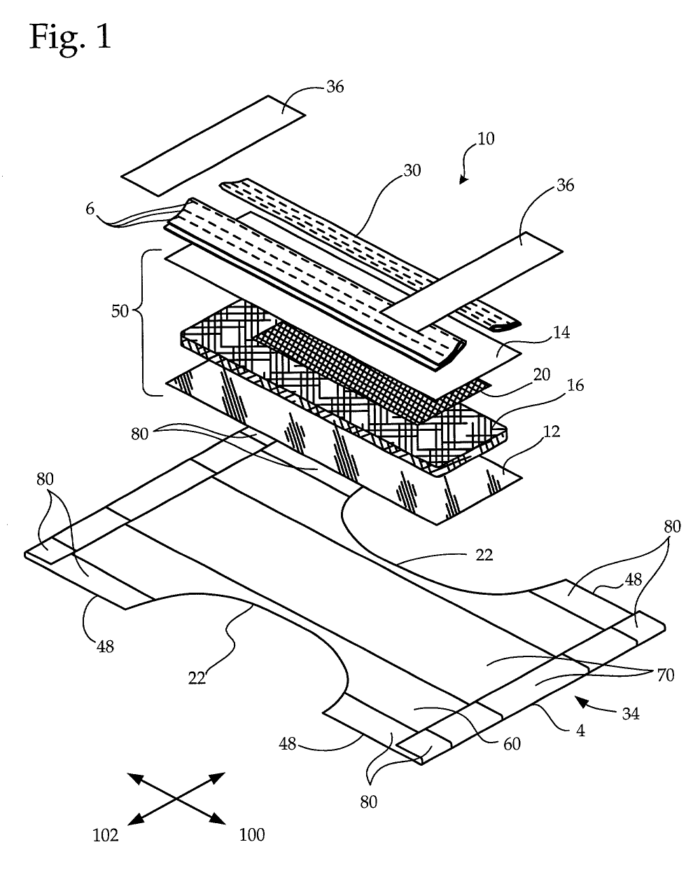

The present invention is described generally with reference to FIG. 1. FIG. 1 is an exploded view of an embodiment of the present invention with the garment laid flat. The garment 10 has a longitudinal axis 100 corresponding approximately to the rear-to-front axis of the wearer, and a lateral axis 102, orthogonal to the longitudinal axis 100, and corresponding approximately to the side-to-side axis of the wearer.

In the embodiment of FIG. 1, the garment 10 preferably comprises a main chassis 34 that forms a pant-like garment 10 having two leg hole cutouts 22 and longitudinal ends 4. The pant-like structure may be formed by joining lateral edge portions 48 to one another to form side seams. The lateral edge portions 48 may be joined during manufacture by any manner known in the art or combinations thereof. Examples of suitable joining mechanisms include: adhesives such as hot melt adhesives and construction adhesives, chemical or solvent bonding, stitching, heat bonding, autogenous bonding, and, preferably, ultrasonic welding. The lateral edge portions 48 also may be joined by a user with the assistance of adhesive strips or mechanical fasteners (not shown). When the lateral edge portions 48 are joined, leg hole cutouts 22 along the lateral edges of the garment 10 form leg holes, and the longitudinal ends 4 of the garment 10 form a waist encircling edge.

A core assembly 50 preferably is disposed on the interior of the chassis 34. The core assembly 50 may comprise an absorbent core 16 disposed between an exterior facing moisture impervious barrier film 12 or “backsheet,” and a moisture pervious body-contacting inner layer 14 or “topsheet.” Each of the backsheet 12, topsheet 14 and absorbent core 16 may comprise a plurality of layers of materials. In the embodiment depicted in FIG. 1, the backsheet 12, topsheet 14, and absorbent core 16 comprise a subassembly that may be attached to the chassis 34. It should be readily apparent that in other embodiments one or both of the topsheet 14 and backsheet 12 may be shaped to form the main body of a pant-like garment thereby eliminating the need for a separate chassis 34. In still another embodiment of the invention, the backsheet 12, topsheet 14, and absorbent core 16 may be assembled and used without ever being shaped as a pant-like garment, such as when used as a feminine care product.

The chassis 34 may comprise a nonwoven polyethylene or polypropylene or any other suitable garment material known in the art or hereafter discovered. All or part of the chassis 34 may comprise a liquid pervious or liquid impervious material or may be zone-treated to be partially liquid pervious or impervious. The chassis 34 may be stretched in one or more directions during the manufacturing process, thereby reducing its elasticity in the direction of stretch.

The backsheet 12 may comprise a laminate of multiple layers of materials that have similar or different properties. The backsheet 12 preferably is made from a substantially liquid impervious material. The selection and manufacture of such materials is well known in the art, and is disclosed, for example, in U.S. Pat. No. 6,123,694 issued to Peniak et al., and U.S. Pat. No. 6,176,952 issued to Maugans et al., each of which is incorporated herein by reference in its entirety, and in a manner consistent with the present invention. In one embodiment, the backsheet 12 is made from a thin thermoplastic material, such as a pigmented polyethylene film having a thickness in the range of 0.02-0.04 mm. The backsheet 12 may also have a laminate construction comprising one or more layers of meltblown polypropylene or meltblown polyethylene, sandwiched between layers of spun-bonded material (often referred to as an “SMS” laminate). Additional layers may be added to the backsheet 12 or the backsheet 12 may be treated with chemicals in order to provide it with other desirable properties, such as to improve the tactile feel, or “hand.” The backsheet 12 may also be entirely or partly gas pervious to allow the garment to circulate air, or “breathe.”

The topsheet 14, which preferably overlays the backsheet 12, can be made from a substantially liquid pervious material to allow body exudates to penetrate into the absorbent core 16. The topsheet 14 may typically comprise a carded polyester fiber with a latex binder or a spun-bonded polypropylene having continuous fibers and thermally bonded by patterned calendar rolls. The topsheet 14 may be treated over all or part of its surface to render it hydrophilic, and may also be zone-treated with a surfactant to render it hydrophilic only in certain target areas. The topsheet 14 also may be treated with skin treating ingredients, such as aloe, vitamin E, and the like, which can be accomplished by a variety of methods known in the art. The topsheet 14 also may comprise an apertured material, such as an apertured film.

In an embodiment of the present invention, one or more of the topsheet 14, backsheet 12 and chassis 34 may comprise a laminate of several layers of material, which may have different physical properties. In another embodiment, one or more of the topsheet 14, backsheet 12 and chassis 34 may comprise several pieces of material, which may have dissimilar physical properties, joined at or near their edges to form a multi-paneled sheet. Such an embodiment is disclosed, for example, in U.S. Pat. No. 5,275,590 issued to Huffman et al., which is incorporated herein by reference in its entirety, and in a manner consistent with the present invention.

In a preferred embodiment of the invention, the topsheet 14 and chassis 34 are comprised of a nonwoven material. The topsheet 14 and backsheet 12 may also be made, however, from any other suitable material. In various embodiments, one or more of the topsheet 14, backsheet 12 and chassis 34 may be selected to provide particular benefits to the garment 10. For example, they may be selected to provide a good tactile impression, or “hand,” a comfortable fit, or gas permeability to improve the breathability of the garment 10.

The absorbent core 16 may be made from any absorbent material or materials known in the art. In one embodiment of the invention, the absorbent core 16 comprises wood fibers or other fibers such as tow fibers, chemical wood pulp, fibrous absorbent gelling material, or any other suitable liquid absorbing material, such as commercially available fluff pulp or fluffed bleached kraft softwood pulp or fibrous absorbent gelling material. In another embodiment of the invention, the absorbent core 16 comprises a combination of a porous fibrous web and super absorbent particles. Absorbent cores are known in the art and are disclosed, for example, in U.S. Pat. Nos. 5,281,207 and 6,068,620 issued to Chmielewski et al., U.S. Pat. No. 4,610,678 issued to Weisman et. al., U.S. Pat. No. 5,137,537 issued to Herron et. al., and U.S. Pat. No. 5,147,345 issued to Young et. al., which are incorporated herein by reference in their entirety, and in a manner consistent with the present invention. In such an embodiment, the absorbent core 16 may be surrounded by a liquid pervious tissue over-wrap (not shown), or other material.

The absorbent core 16 generally is elongated along the longitudinal axis 100 of the garment, and may extend along either or both of the lateral and longitudinal axes 102, 100 to the outer perimeter of the garment. In the embodiment depicted in FIG. 1, the absorbent core 16 is substantially rectangular in shape, however, it may also have rounded ends or other shapes, such as an “I” shape or a “T” shape. The absorbent core 16 also may have channels, grooves or pockets, and may have a varying thickness.

The various parts of the garment 10 preferably are operatively associated with one another in such a manner that the garment will maintain its desired structure during use. The parts may be operatively associated with one another by a variety of methods known in the art, including, but not limited to: using adhesives such as hot melt adhesives and construction adhesives, chemical or solvent bonding, ultrasonic welding, stitching, heat bonding, autogenous bonding, or any other method of affixation known or hereafter discovered. U.S. Pat. No. 4,919,738 issued to Ball et. al. discloses a method of autogenous bonding, and its disclosure is herein incorporated by reference in its entirety in a manner consistent with the invention. All of the parts may be joined to each adjacent part, but some parts may not be joined to others. In one embodiment, the topsheet 14 and backsheet 12 are bonded to one another around their perimeter regions, thereby encasing and holding the absorbent core 16 in place without having to directly join the absorbent core 16 to any other component parts of the garment 10. The topsheet 14 or backsheet 12 may also be operatively associated with the absorbent core 16. As understood herein, the term “operatively associated” includes directly joining one part to another, indirectly joining parts together through one or more intermediary parts, whether those intermediary parts are described herein or not, joining parts in such a manner that unjoined parts are captured or held in their proper place, and any other suitable joining means that maintains the structural integrity of the garment 10 for the duration of its use.

In the embodiment of the invention depicted in FIG. 1, the garment 10 further comprises various mechanisms for improving the garment's ability to contain body exudates, such as standing leg gathers 30. Standing leg gathers 30 may be formed by incorporating a plurality of gather elastics 6 into folds in the topsheet 14 or into additional ribbons that are attached to the garment near the leg hole cutouts 22. The gather elastics 6 cause the standing leg gathers 30 to rise above the interior surface of the garment 10, thereby forming vertical curtains of material that help contain exudates. The ribbons may be liquid pervious or liquid impervious, and more than one set of standing leg gathers 30 may be provided. The standing leg gathers 30 may be attached to the topsheet 14, backsheet 12, chassis 34 or any other suitable part of the garment such that they block or impede the passage of fluids and other exudates. Additional elastics (not shown) may also be incorporated into the chassis 34, topsheet 14 or backsheet 12 adjacent the leg holes to form conventional (i.e., non-standing) leg gathers, as is known in the art. Conventional gathers contract the garment 10 around the wearer's legs and body to prevent leakage. U.S. Pat. Nos. 3,860,003 and 4,081,301 issued to Buell, U.S. Pat. No. 4,695,278 issued to Lawson, U.S. Pat. No. 4,808,177 issued to Des Marais, U.S. Pat. No. 4,795,454 issued to Dragoo, and U.S. Pat. No. 4,938,755 issued to Foreman illustrate other embodiments of leg cuffs and gathers in absorbent garments, and the disclosures of each of these patents are hereby incorporated by reference in their entirety.

The core assembly 50 may comprise additional layers of material that may reduce rewet of the topsheet 14, reduce strikethrough times or otherwise improve the absorbency, dryness and other properties of the garment 10. For example, a transfer layer 20 comprising an apertured film, a foam material or an air-bonded carded, bicomponent fiber nonwoven, having a basis weight of about 40 g/m² may be disposed between the topsheet 14 and the absorbent core 16. Such multiple layer absorbent cores are known in the art and disclosed in U.S. Pat. No. 5,439,458 issued to Noel et al., which is incorporated herein by reference in its entirety, and in a manner consistent with the present invention.

The core assembly 50 may be attached to the chassis 34 by any manner known in the art, such as by ultrasonic bonding or by the use of lines of hot melt adhesive. The bond between the core assembly 50 and the chassis 34 may be reinforced by laterally-extending end strips 36 that are applied over the longitudinal ends of the core assembly 50 and bonded to the underlying structure of the garment 10. The end strips 36 also may hold the ends of the standing leg gathers 30. Such end strips 36 preferably comprise a fluid pervious nonwoven material, but may be fluid impervious or a material other than a nonwoven material. Such materials are known in the art. The end strips 36 also may help prevent the longitudinal flow of exudates past the ends of the core assembly 50, particularly if the edges of the nonwoven strips overlying the core assembly 50 are left unbonded so that they form pockets to hold exudates.

In other embodiments, adjustment strips (not shown) may be disposed on and partially attached to the garment to provide for an adjustable fit. Absorbent garments often loosen during use for various reasons, such as inelastic stretching of the various components, changes in user size, and increased loading caused by the release of body exudates into the garment 10. The adjustment strips may be formed such that they may be releasably attached to the garment 10 to reduce the circumference of the waist encircling edge, and may comprise any fastening mechanism known in the art or later discovered.

It often is desirable for an absorbent garment to contract around various parts of the wearer's body to provide improved comfort and exudate containment. In addition to the standing leg gathers 30 or conventional gathers, waist elastics and tummy elastics may be incorporated into the garment 10 to contract the garment 10 about the wearer's waist and stomach. Such elastics are typically stretched as they are joined to the garment 10 so that the contraction of the elastics causes the garment 10 to contract about the wearer. The elastics may also be applied in an unstretched stated then mechanically stretched to create an elasticized region in the garment (often called a zero-strain laminate). The elastics also may be applied in an inelastic state then heat activated to cause them to be come elasticized. The elastics may be made from natural or synthetic rubber, elastomers, LYCRA® elastomer (available from E. I. DuPunt de Nemours and Company, a business having offices in Wilmington, Del.), polyurethane, heat shrinkable polymer ribbons, or any other suitable elastic material or composite.

FIG. 1 is an example of a garment in accordance with particularly preferred embodiments of the invention. In FIG. 1, elastic layer 60 is a part of chassis 34 and has substantially the same area as chassis 34. In this example, central inelastic portion 70 is attached to elastic layer 60 in a central region of the garment extending along the longitudinal axis 100. While central inelastic portion 70 is shown as continuing all the way to longitudinal ends 4 (including the fold that exists at longitudinal ends 4), it is noted that central inelastic portion 70 can also terminate short of longitudinal ends 4. The width of central inelastic portion 70 can be, as shown in the example, uniform or it can change at different positions along longitudinal axis 100. In this example, the width (the dimension along lateral axis 102) of central inelastic portion 70 is set to be approximately equal to the width of backsheet 12 so that backsheet 12 can be bonded to central inelastic portion 70 effectively. This construction aids in the effective bonding of backsheet 12 to chassis 34 because an inelastic material, which backsheet 12 typically is, will usually bond much more effectively to another inelastic material than to an elastic material.

FIG. 1 also shows side inelastic portions 80 located near lateral edge portions 48 of chassis 34. As with central inelastic portion 70, side inelastic portions 80 provide a region of chassis 34 that is inelastic and, therefore, a desirable region for bonding. In the case of a garment that is assembled into a pant type garment by the manufacturer, side inelastic portions 80 provide excellent regions for bonding to each other to form the side seams of the pant type garment. In the case of a diaper type garment that has side connections that are made by a caregiver of the user, side inelastic portions 80 provide excellent regions for bonding diaper closing mechanisms such as hook and loop fasteners or adhesive tape to chassis 34.

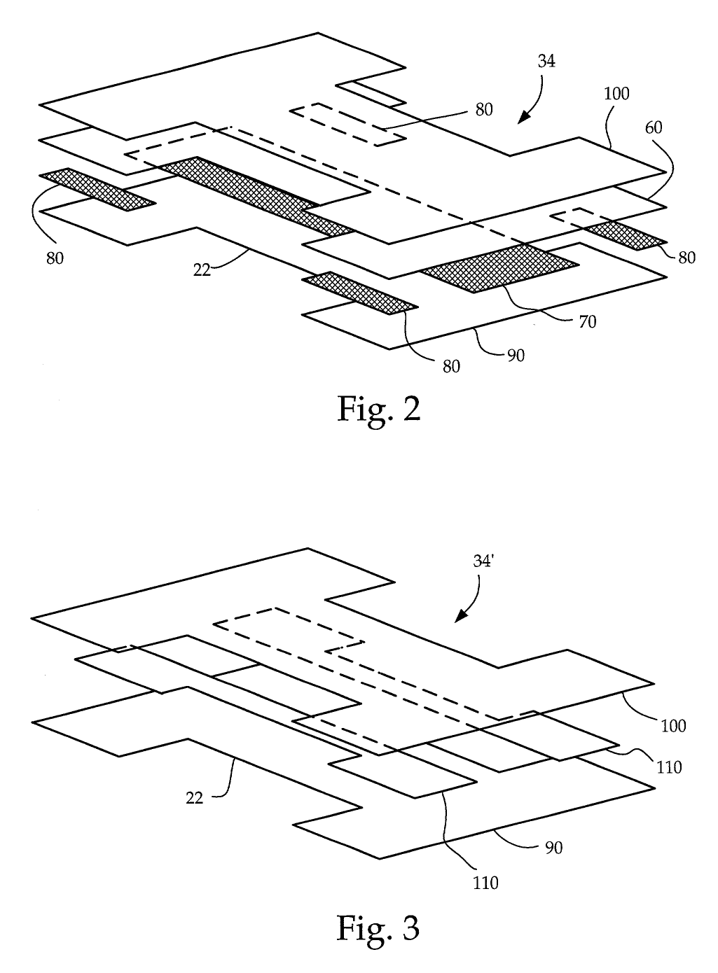

FIG. 2 shows an exploded view of the embodiment of chassis 34 shown in FIG. 1. FIG. 2 shows a preferred laminate composition of chassis 34 that includes elastic layer 60, central inelastic portion 70 and side inelastic portions 80 captured between a first layer 90 and a second layer 100. In this embodiment, elastic layer 60 has an overall area substantially equal to that of first layer 90 and second layer 100. Central inelastic portion 70 and side inelastic portions 80 are located in just those areas of chassis 34 that elasticity is not desired. Although central inelastic portion 70 and side inelastic portions 80 are shown in FIG. 2 as being located between elastic layer 60 and first layer 90, it is noted that central inelastic portion 70 and side inelastic portions 80 could be, instead, located at any other position within the laminate. In addition, it may be preferable to omit either first layer 90 or second layer 100 from the laminate.

FIG. 3. shows another embodiment of the invention that provides the desired inelastic portions. In the embodiment shown in FIG. 3, central inelastic portion 70 and side inelastic portions 80 are not used. Instead, elastic layer 60 is replaced with elastic portions 110 having a total area less than that of chassis 34′. Although FIG. 3 shows no central inelastic portion or side inelastic portions, it is noted that it may be preferable to add inelastic portions in particular areas to the embodiment shown in FIG. 3 to provide inelastic regions within, for example, the elastic portions 110. In addition, inelasticity may be imparted to chassis 34 by use of first or second layers 90, 100, that are inelastic.

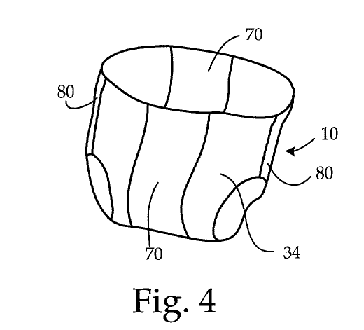

FIG. 4 illustrates the embodiment shown in FIG. 1 in an assembled condition (with various components omitted for clarity). FIG. 4 illustrates how the two-side inelastic portions 80 adjacent to each leg hole cutout 22 are adjacent to each other in the assembled condition.

While the invention has been described with reference to particular embodiments and examples, those skilled in the art will appreciate that various modifications may be made thereto without significantly departing from the spirit and scope of the invention.

I claim:

1. An absorbent garment, comprising:
a main chassis having an attachment region;
an absorbent portion operatively associated with the main chassis at the attachment region; and
an elastic portion operatively associated with the main chassis,
wherein the attachment region is substantially inelastic prior to the absorbent portion being operative associated with the main chassis.

2. The garment of claim 1, wherein the attachment region comprises no elastic material.

3. The garment of claim 2, wherein the main chassis further comprises a dead region, the dead region comprising no elastic material.

4. The garment of claim 3, wherein the dead region comprises four dead regions,
the garment has four garment-joining regions at which the garment is attached to itself to create a wearable garment, and
each of the four dead regions is located at one of the four garment-joining regions.

5. The garment of claim 1, wherein the attachment region comprises a laminate having
an elastic layer; and
a substantially inelastic layer.

6. The garment of claim 5, wherein the attachment region further comprises a first layer of nonwoven material.

7. The garment of claim 6, wherein the attachment region further comprising a second layer of nonwoven material, and
the elastic layer and the substantially inelastic layer are positioned between the first and second layers of nonwoven material.

8. The garment of claim 7, wherein the absorbent portion is attached directly to the attachment region.

9. The garment of claim 5, wherein the main chassis further comprises a dead region, the dead region being substantially inelastic.

10. The garment of claim 9, wherein the dead region comprises four dead regions,
the garment has four garment-joining regions at which the garment is attached to itself to create a wearable garment, and
each of the four dead regions is located at one of the four garment-joining regions.

11. The garment of claim 10, wherein the elastic layer is substantially equal in area to the main chassis.

12. The garment of claim 11, wherein the substantially inelastic layer comprises a band of inelastic material having
a first end located substantially at a front waist edge of the garment, the front waist edge being located proximate a belly of a user of the garment, and
a second end located substantially at a back waist edge of the garment, the back waist edge being located proximate a back of the user of the garment.

13. The garment of claim 12, wherein a longitudinal direction is a direction from the front waist edge to the back waist edge that passes through a crotch region of the garment,
a transverse direction is a direction perpendicular to the longitudinal direction, and
the band of inelastic material has a width in the transverse direction at least as great as a width of the absorbent portion in the transverse direction.

13. The garment of claim 5, wherein the elastic layer is substantially equal in area to the main chassis.

14. The garment of claim 13, wherein the substantially inelastic layer comprises a band of inelastic material having
a first end located substantially at a front waist edge of the garment, the front waist edge being located proximate a belly of a user of the garment, and
a second end located substantially at a back waist edge of the garment, the back waist edge being located proximate a back of the user of the garment.

15. An absorbent garment main chassis, comprising:
a first carrier layer;
an elastic layer; and
a substantially inelastic layer,
wherein the elastic layer is substantially equal in area to the first carrier layer,
the substantially inelastic layer is smaller in area than the first carrier layer, and
the substantially inelastic layer is selectively located to create areas of inelasticity.

16. The chassis of claim 15, further comprising a second carrier layer,
wherein at least one of the elastic layer and the substantially inelastic layer is disposed between the first carrier layer and the second carrier layer.

17. The chassis of claim 15, wherein the substantially inelastic layer comprises a strip positioned along a longitudinal axis of the chassis extending from a location substantially at a front waist edge, through a crotch portion, and to a location substantially at a back waist edge.

18. The chassis of claim 17, wherein the substantially inelastic layer further comprises a plurality of garment joining region portions, each of which is located on the main chassis at a location corresponding to a garment joining region of the chassis.

19. The chassis of claim 18, further comprising a second carrier layer,
wherein at least one of the elastic layer and the substantially inelastic layer is positioned between the first carrier layer and the second carrier layer.
